SEQUENCE LISTING (4 sequences)

SEQ ID NO: 1 - protein, 229 aa, Vesicular stomatitis virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 2 - DNA, 690 bp, Vesicular stomatitis virus
atgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatga

SEQ ID NO: 3 - protein, 229 aa, Vesicular stomatitis virus
MSSLKKILGLKGKGKKSKKLGIAPPPYEEDTSMEYAPSAPIDKSYFGVDERDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWFLDSIRHFK

SEQ ID NO: 4 - DNA, 690 bp, Vesicular stomatitis virus
atgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagagggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtggttcctggactctatccggcacttcaaatga

(19) United States
(12) Patent Application Publication
(10) Pub. No.: US 2022/0296659 A1
(43) Pub. Date: Sep. 22, 2022

(54) PHARMACEUTICAL COMPOSITION FOR TREATMENT OF TUMOR OR CANCER, AND APPLICATION THEREOF

(71) Applicant: FANTASIA BIOPHARMA (ZHEJIANG) CO. LTD, Jinhua, Zhejiang (CN)

(72) Inventor: Frank Xiaofeng QIN, Jinhua (CN)

(73) Assignee: FANTASIA BIOPHARMA (ZHEJIANG) CO. LTD, Jinhua, Zhejiang (CN)

(21) Appl. No.: 17/611,726

(22) PCT Filed: Dec. 6, 2019
(86) PCT No.: PCT/CN2019/123567

§ 371 Date: May 31, 2022

(30) Foreign Application Priority Data
May 17, 2019 (CN) 2019 10413002.6

(51) Int. Cl. A61K 35/766; A61K 31/192; A61K 45/06; A61P 35/00; A61P 11/00; A61P 35/04

(52) U.S. Cl. CPC A61K 35/766; A61K 31/192; A61K 45/06; A61P 35/00; A61P 11/00; A61P 35/04

(57) ABSTRACT

Provided are a pharmaceutical composition for treatment of a tumor or cancer, and an application thereof. The pharmaceutical composition comprises an oncolytic rhabdovirus and a small molecule CD38 inhibitor such as rhein that are administered via direct local injection or systemic administration or intratumoral delivery. The oncolytic rhabdovirus has the characteristic of recognizing tumor cells and would not cause damage to normal cells. Meanwhile, the small molecule CD38 inhibitor has the activity of specifically inhibiting T-cell receptor molecules. The combined use of the oncolytic rhabdovirus and the small molecule CD38 inhibitor has significant advantages in safety and efficacy.

### SEQUENCE LISTING

The present application contains a Sequence Listing that has been submitted electronically in ASCII format and is hereby incorporated by reference in its entirety. The ASCII copy, created on May 31, 2022, is named Substitute Sequence Listing_ST25.txt and is 8,192 bytes in size.

### TECHNICAL FIELD

The present disclosure belongs to the field of biotechnology, and specifically relates to a pharmaceutical composition and its use in drugs for treating tumor or cancer.

### BACKGROUND

Malignant tumors are the main diseases that cause the death of human, and the main therapeutic methods are surgery, radiotherapy and chemotherapy. Biotherapy has been developed in recent years and is referred to as the fourth method for treating malignant tumors. It includes tumor vaccine therapy, tumor non-specific immunotherapy, antibody immunotherapy, cytokine therapy, adoptive cell immunotherapy, tumor gene therapy, etc. Tumor arises from the accumulated changes in gene and epigenetics in normal cells, and such changes drive the transformation of normal cells into malignant tumors. This complex process of the pathological changes determines the diversity of mechanisms in the occurrence, maintenance and metastasis of different tumors. Currently, surgical resection, chemotherapy and radiotherapy are methods commonly used for clinical treatment of tumors, however, tumors are prone to recur after the surgical resection of tumors, and the toxic and side effects of radiotherapy and chemotherapy are relatively large.

The method of treating cancer with virus also belongs to biotherapy and has developed rather rapidly in the past two decades. Currently, one of the greatest progresses in viral gene therapy is optimizing and modifying the structures of some viruses by utilizing the difference between tumor cells and normal cells, so as to enable these viruses to replicate in tumor cells selectively, thereby achieving the purpose of killing tumor cells finally. These modified viruses are collectively referred to as oncolytic viruses according to their functions, and their origins include but are not limited to adenovirus, vesicular stomatitis virus, herpes virus, poxvirus, and the like. Currently, it has been found that some wild-type viruses also have the function of replicating in tumor cells selectively and causing oncolysis.

The component in an oncolytic rhabdovirus injection launched in China is a genetically modified adenovirus type 5 (H101), which facilitates the replication of virus in tumor cells. Principally, E1B-55KD gene segment and E3 gene segment of human adenovirus type 5 are deleted in H101, and H101 has the characteristic of specifically replicating in tumor cells and eventually leading to oncolysis. H101 replicates abundantly in tumor cells after administered via intratumoral injection, thereby resulting in the lysis and death of tumor cells eventually. The component in an oncolytic rhabdovirus (T-Vec) approved by the USFDA is a genetically engineered and modified herpes simplex virus type 1 (HSV-1). In T-Vec, ICP34.5 gene and ICP47 gene are deleted and the gene of granulocyte-macrophage colony-stimulating factor (GM-CSF) which is a protein capable of activating human immune system is inserted, and T-Vec is capable of replicating in tumor cells and expressing GM-CSF. Direct injection of T-Vec into melanoma lesions may cause the lysis of tumor cells, thereby rupturing the tumor cells, enabling the tumor cells to release tumor-derived antigens and GM-CSF and accelerating the antitumor immune response. However, according to Amgen, the exact mechanism of this effect is still unclear. On Oct. 27, 2015, T-Vec was approved by FDA as a local therapeutic regimen for unresectable lesions in patients that suffer from melanoma and have experienced recurrence after the first surgery.

Oncolytic viruses are a class of replicable viruses that targetedly infect and kill tumor cells without destroying normal cells. Oncolytic virotherapy is an innovative tumor-targeted therapeutic strategy, which uses natural viruses or genetically engineered and modified viruses to infect tumor cells selectively and allows such viruses to replicate in tumor cells, thereby achieving the effect of targeted lysis and killing of tumor cells without causing harm to normal cells.

However, oncolytic virotherapy mainly faces the following two problems. First, the antitumor spectrum of oncolytic virus is relatively narrow. To be specific, tumor cells that are sensitive to oncolytic viruses are accompanied by more virus replication, while tumor cells that are not sensitive to oncolytic viruses are accompanied by less virus replication. Therefore, there is a need to screen out oncolytic virus strains with broad-spectrum infection efficiency. Secondly, the replication of a virus would be restricted over time in vivo and the virus would be slowly eliminated by the body. Therefore, how to selectively and effectively increase the replication of oncolytic viruses in tumor cells is an urgent problem to be solved.

Rhein is abundant in the rhizome of rhubarb which is a Chinese herbal medicine, and is an anthraquinone-based compound. In traditional Chinese medicine theory, rhein has purgative effect, diuretic effect and bacteriostatic effect. However, with the development of modern Chinese medicine theory, it has been found that rhein also has immunosuppressive effect, regulative effect of in-vivo metabolism and antitumor effect. Among them, the antitumor effect has been a research hotspot in recent years. It has been found by research that rhein has a certain inhibitory effect on melanoma cells, Ehrlich ascites cancer cells, liver cancer cells, breast cancer cells and P388 leukemia cells of mice, and experiments using rhein for the treatment of other tumors are being gradually carried out. The antitumor mechanisms of rhein are mainly classified into three aspects. First, rhein is capable of influencing the cell proliferation kinetics of tumor cells. Kuo et al. has confirmed that rhein is capable of arresting the cell cycle by increasing the expression of P53 protein and P21/WAF1 protein. In the second place, rhein is also capable of inhibiting the binding of the cyclin-dependent regulatory subunits to the catalytic subunits at the end of G phase, so as to make p34cdc2 protease inactive and not capable of positively regulating the S phase-specific promoter, thereby inhibiting the transition from G1 phase to S phase. In addition, the amount of hypodiploid cells and fragmented DNA is increased, thereby inducing cell apoptosis. Studies have also confirmed that rhein may bind to AP-1 as a substrate, thereby increasing the sensitivity of cells to cytotoxic agents and being capable of inducing cell apoptosis in a case where no DNA is inserted. Secondly, rhein may influence the energy metabolism of tumor cells. Rhein reacts with glutamic acid (acting as a substrate) in liver cells, which reduces the mitochondrial membrane potential, inhibits the electron transport in the respiratory chain and leads to the death of mitochondria, thereby causing cell apoptosis. In the second place, rhein may also lead to relatively less protein synthesis by affecting cellular respiration and glycolysis, thereby reducing the survival rate of cells. Thirdly, rhein has antimutagenic effect. Cytochrome P50 (CYP1A1) is a carcinogenic-related metabolic enzyme, and it has been confirmed by experiments that rhein is capable of regulating the activity of CYP1A1 by inhibiting the induction of CYP1A1 caused by the carcinogen 3-amino-1-methyl-5H-pyridox[4,3,b]indole (Trp-P-2). In addition, rhein may also inhibit the activation of the transcription factor AP-1 and the cell transformation induced by the cancer promoter TPA.

Currently, rhein has poor therapeutic effect on tumor cells in vivo when used alone, and is mostly used in combination with other drugs. The combination with vitamin C and E is capable of increasing the toxicity to colon cancer cells in vivo. The combined use of rhein and mitomycin (MMC) is capable of inhibiting the transmembrane transport of nucleosides in tumor cells, inducing the apoptosis of KB cells and significantly enhancing the inhibitory effect of MMC on the proliferation of tumor cells. In addition, when rhein and doxorubicin are used in combination to treat human neuroglioma, rhein inhibits the reduction of ferricyanide dose-dependently. Ferricyanide induces the release of protons, inhibits the synthesis of ATP and also inhibits the membrane redox system, thus resulting in the decrease of cell viability. Moreover, these two drugs have a strong synergistic effect and may act on different targets. As a result, a low dose of ADM is capable of exerting an inhibitory effect, thereby increasing the therapeutic index of ADM and reducing the toxicity of ADM to normal cells.

However, there is still room for improvement in the prior art for the time being. First, the overall response rate of rhein is not high. In the second place, currently, the effective control rate of an immunotherapy drug is approximately 10% in tumors such as melanoma and colon cancer when used alone, and is not a very high response rate as compared with traditional therapy, therefore, an immunotherapy drug alone cannot be formulated into a medicine. As a result, there is still a need for more effective therapeutic regimens for tumors and drugs or compositions based on the aforementioned therapeutic regimens.

### SUMMARY

### Problems to be Solved by the Disclosure

In order to solve the above-mentioned problems existing in the prior art, the present disclosure provides a composition capable of improving the overall control rate of tumor patients and further improving the cure rate of tumor patients, as well as the use of the aforementioned composition.

### Means for Solving the Problems

The technical solutions adopted in the present disclosure are as follows.

In one technical solution, the present disclosure provides a composition, wherein the composition comprises (a) an oncolytic rhabdovirus and (b) a CD38 molecule inhibitor.

In the composition according to the present disclosure, the oncolytic rhabdovirus is a vesicular stomatitis virus or a Maraba virus, or a recombinant vesicular stomatitis virus or a recombinant Maraba virus retaining the biological activity of the vesicular stomatitis virus or the Maraba virus; preferably, the vesicular stomatitis virus is selected from Indiana Strain of vesicular stomatitis virus, Nancy strain of vesicular stomatitis virus, and MuddSummer strain of vesicular stomatitis virus; more preferably, the recombinant vesicular stomatitis virus is selected from recombinant virus strains of said MuddSummer strain of vesicular stomatitis virus; and

alternatively, the recombinant vesicular stomatitis virus or the recombinant Maraba virus has oncolytic and/or attenuated activity as compared with the corresponding wild-type virus.

In the composition according to the present disclosure, the oncolytic rhabdovirus comprises a modified matrix protein (M), an amino acid sequence encoding the modified matrix protein (M) has at least 80%, preferably at least 90%, more preferably at least 95%, and most preferably at least 98% identity with the amino acid sequence as set forth in SEQ ID NO: 1; and

the amino acid sequence has amino acid substitutions at position 51, position 221 and position 226 as compared with SEQ ID NO: 1.

In the composition according to the present disclosure, the sequence of the modified matrix protein (M) is the amino acid sequence encoding the modified matrix protein (M) and has the following mutations as compared with SEQ ID NO:1:

(i) mutation of methionine M to arginine R at position 51,

(ii) mutation of valine V to phenylalanine F at position 221, and

(iii) mutation of glycine G to arginine R at position 226,

preferably, the sequence of the modified matrix protein (M) is a sequence as set forth in SEQ ID NO:3.

In the composition according to the present disclosure, the CD38 molecule inhibitor is selected from a combination comprising one or more selected from rhein and its analogs; preferably, the CD38 molecule inhibitor is selected from rhein, a physiologically or pharmaceutically acceptable salt or ester thereof, or a combination thereof.

In the composition according to the present disclosure, the active substances in the composition further comprise one or more selected from a combination of other active substances that control or treat a tumor, wherein said other active substance is selected from clofibrates, choline, methionine, niacin-based substances and ursodeoxycholic acid.

In the composition according to the present disclosure, the composition further comprises a second oncolytic virus; preferably, the second oncolytic virus is one or more selected from the group consisting of a rhabdovirus, a vaccinia virus, a herpes virus, a measles virus, a Newcastle disease virus, an adenovirus, an alphavirus, a parvovirus, and an enterovirus strain; more preferably, the second oncolytic virus is an attenuated oncolytic virus; and most preferably, the second oncolytic virus is an attenuated rhabdovirus.

In the composition according to the present disclosure, the composition further comprises a second antitumor preparation; preferably, the second antitumor preparation is an immunotherapeutic agent, a chemotherapeutic agent or a radiotherapeutic agent; and more preferably, the second antitumor preparation is one or more selected from the group consisting of small molecules, macromolecules, cells, viral vectors, gene vectors, DNAs, RNAs, polypeptides and nanocomposites.

In the composition according to the present disclosure, the composition comprises the oncolytic rhabdovirus and the CD38 molecule inhibitor of a single administration dose, the oncolytic rhabdovirus has a single administration dose ranging from 1×10⁵ PFU to 1×10¹¹ PFU, and the CD38 molecule inhibitor has a single administration dose ranging from 10 to 50 mg/kg.

In the composition according to the present disclosure, a single administration dose of the oncolytic rhabdovirus is 1×10⁷ PFU of virus per tumor volume of 100 mm³, and the CD38 molecule inhibitor has a single administration dose of 10 mg/kg.

In the composition according to the present disclosure, the oncolytic rhabdovirus and the CD38 molecule inhibitor are each independently present in the composition without being mixed with each other.

In the composition according to the present disclosure, the oncolytic rhabdovirus is a genetically mutated attenuated strain having oncolytic effect or a wild-type virus having oncolytic effect; preferably, the oncolytic rhabdovirus is an attenuated strain of vesicular stomatitis virus having targeted oncolytic effect or an attenuated strain of Maraba virus having targeted oncolytic effect.

In another technical solution, the present disclosure provides a use of the composition according to the present disclosure in preparation of a drug for killing abnormally proliferating cells, inducing and promoting antitumor immune response or eliminating immunosuppression in a microenvironment of a tumor tissue.

In the use according to the present disclosure, the composition comprises a clinically administered dose of the oncolytic rhabdovirus, the oncolytic rhabdovirus has a single administration dose ranging from 1×10⁵ PFU to 1×10¹¹ PFU, and the CD38 molecule inhibitor has a single administration dose ranging from 10 to 50 mg/kg; preferably, the oncolytic rhabdovirus has a single administration dose of 1×10⁷ PFU, and the CD38 molecule inhibitor has a single administration dose of 10 mg/kg.

In the use according to the present disclosure, the abnormally proliferating cells are contained in the body of a patient; alternatively, the abnormally proliferating cells are selected from tumor cells and tumor tissue-related cells; preferably, the tumor cells are cancer cells; and more preferably, the cancer cells are metastatic cancer cells.

In another technical solution, the present disclosure provides a use of the composition according to the present disclosure in preparation of a drug for treating a patient suffering from tumor and/or cancer.

In the use according to the present disclosure, the composition comprises a clinically administered dose of the oncolytic rhabdovirus, the oncolytic rhabdovirus has a single administration dose ranging from 1×10⁵ PFU to 1×10¹¹ PFU, and the CD38 molecule inhibitor has a single administration dose ranging from 10 to 50 mg/kg; preferably, the oncolytic rhabdovirus is administered to a tumor with a volume of 100 mm³ at a single administration dose of 1×10⁷ PFU, and the CD38 molecule inhibitor has a single administration dose of 10 mg/kg.

In another technical solution, the present disclosure also provides a method for inhibiting and/or killing abnormally proliferating cells in a subject, the method comprising sequentially carrying out the following steps in the subject:

1) administering an oncolytic rhabdovirus to the subject, wherein the oncolytic rhabdovirus is capable of replicating in tumor cells selectively; and

2) administering a CD38 molecule inhibitor to the subject after administration of the oncolytic rhabdovirus in step 1);

alternatively, administering the CD38 molecule inhibitor to the subject 24 hours to 48 hours after the administration of the oncolytic rhabdovirus.

In the method according to the present disclosure, the oncolytic rhabdovirus is the oncolytic rhabdovirus, and the CD38 molecule inhibitor is selected from a combination comprising one or more selected from rhein and its analogs; more preferably, the CD38 molecule inhibitor is selected from rhein, a physiologically or pharmaceutically acceptable salt or ester thereof, and a combination thereof.

In the method according to the present disclosure, the oncolytic rhabdovirus is a clinically administered dose of the oncolytic rhabdovirus, the oncolytic rhabdovirus has a single administration dose ranging from 1×10⁵ PFU to 1×10¹¹ PFU, the CD38 molecule inhibitor is a clinically administered dose of the CD38 molecule inhibitor, and the CD38 molecule inhibitor has a single administration dose ranging from 10 to 50 mg/kg; preferably, the oncolytic rhabdovirus has a single administration dose of 1×10⁷ PFU per tumor volume of 100 mm³, and the CD38 molecule inhibitor has a single administration dose of 10 mg/kg.

In the method according to the present disclosure, the administration dose of the oncolytic rhabdovirus is the clinically administered dose and the oncolytic rhabdovirus is administered once every three days for three consecutive times; and the rhein is administered once every two days for three to five consecutive times.

In the method according to the present disclosure, the oncolytic rhabdovirus, a composition comprising an isolated recombinant oncolytic rhabdovirus, or a vaccine comprising an isolated recombinant oncolytic rhabdovirus is administered via one or more administration modes selected from the group consisting of intraperitoneal administration, intravenous administration, intraarterial administration, intramuscular administration, intradermal administration, intratumoral administration, subcutaneous administration and intranasal administration; preferably, administration routes of the administration modes include one or more selected from the group consisting of endoscopy, celioscopy, intervention, minimal invasive surgery and traditional surgery; alternatively, said rhein is administered via intravenous administration or intraperitoneal administration.

In the method according to the present disclosure, the abnormally proliferating cells is selected from tumor cells and/or cancer cells.

The method according to the present disclosure further comprises a step of administering a second antitumor therapy.

In the method according to the present disclosure, the second antitumor therapy is administering a second oncolytic virus; preferably, the second oncolytic virus is one or more selected from the group consisting of a rhabdovirus, a vaccinia virus, a herpes virus, a measles virus, a Newcastle disease virus, an adenovirus, an alphavirus, a parvovirus, and an enterovirus strain; more preferably, the second oncolytic virus is an attenuated oncolytic virus; and most preferably, the second oncolytic virus is an attenuated oncolytic rhabdovirus.

In the method according to the present disclosure, the tumor and/or cancer is selected from lung cancer, melanoma, head and neck cancer, liver cancer, brain cancer, colorectal cancer, bladder cancer, breast cancer, ovarian cancer, uterine cancer, cervical cancer, lymphoma, gastric cancer, esophageal cancer, kidney cancer, prostate cancer, pancreatic cancer, and leukemia.

In the method according to the present disclosure, the second antitumor therapy is one or more selected from the group consisting of chemotherapy, radiotherapy, immunotherapy and surgical therapy.

In another technical solution, the present disclosure also provides a method for inducing immune response in a subject, wherein the method comprises administering the composition of the present disclosure to the subject.

In the method according to the present disclosure, the oncolytic rhabdovirus in the composition is the oncolytic rhabdovirus, and the CD38 molecule inhibitor in the composition is selected from a combination comprising one or more selected from rhein and its analogs; preferably, the CD38 molecule inhibitor is selected from rhein, a physiologically or pharmaceutically acceptable salt or ester thereof, and a combination thereof.

The method according to the present disclosure comprises sequentially carrying out the following steps in the subject:

1) administering an oncolytic rhabdovirus to the subject, wherein the oncolytic rhabdovirus is capable of replicating in tumor cells selectively; and

2) administering a CD38 molecule inhibitor to the subject after administration of the oncolytic rhabdovirus in step 1);

alternatively, administering the CD38 molecule inhibitor to the subject 24 hours to 48 hours after the administration of the oncolytic rhabdovirus.

In another technical solution, the present disclosure also provides a method for inducing and promoting antitumor immune response or eliminating immunosuppression in a microenvironment of a tumor tissue, wherein the method comprises a step of contacting a tumor or a tumor tissue with the composition of the present disclosure.

In the method according to the present disclosure, the oncolytic rhabdovirus is the oncolytic rhabdovirus, and the CD38 molecule inhibitor is selected from a combination comprising one or more selected from rhein and its analogs; preferably, the CD38 molecule inhibitor is selected from rhein, a physiologically or pharmaceutically acceptable salt or ester thereof, and a combination thereof.

The method according to the present disclosure comprises the following steps:

1) administering an oncolytic rhabdovirus to the subject so as to allow the tumor or the tumor tissue of the subject to contact with the oncolytic rhabdovirus, wherein the oncolytic rhabdovirus is capable of replicating in tumor cells selectively; and

2) after administration of the oncolytic rhabdovirus in step 1), administering a CD38 molecule inhibitor to the subject so as to allow the tumor or the tumor tissue of the subject to contact with the CD38 inhibitor;

alternatively, administering the CD38 molecule inhibitor to the subject 24 hours to 48 hours after the administration of the oncolytic rhabdovirus.

### Advantageous Effects of the Disclosure

In one embodiment, the present disclosure combines small molecule inhibitor therapy with broad-spectrum oncolytic virotherapy, so as to improve the overall response rate and the cure rate of tumor patients.

In one embodiment, the present disclosure combines a small molecule inhibitor with specificity and an oncolytic virotherapy with specificity, so as to improve the overall control rate of tumor patients and further improve the cure rate of tumor patients. In a specific embodiment, the small molecule inhibitor with specificity is selected from CD38 inhibitors. In another specific embodiment, the small molecule inhibitor with specificity is rhein.

In one embodiment, the present disclosure provides a combination therapeutic regimen of an oncolytic virus and a small molecule CD38 inhibitor, which is used for inhibiting and/or killing abnormally proliferating cells.

In one embodiment, the composition provided in the present disclosure is a combination of an attenuated virus U400 targeting the tumor microenvironment and rhein. The aforementioned oncolytic virus U400 is capable of changing the tumor microenvironment effectively and facilitating the effective infiltration of autologous immune cells into local tumor tissue. Rhein breaks the inhibitory effect on T cells exerted by tumor cells and facilitates the specific killing effect of CTL cells. Meanwhile, the combined use of drugs significantly improves the ability to track and kill metastatic tumor cells and significantly increases the control rate of metastatic tumors.

### BRIEF DESCRIPTION OF THE DRAWINGS

FIG. 1 shows a schematic diagram illustrating the development of metastatic lung cancer model.

FIG. 2 shows the evaluation of the therapeutic effects of different mutant strains in a transplanted tumor model. Among them, FIG. 2A is a schematic diagram of the inoculation procedure of LLC lung cancer cells, and FIG. 2B to FIG. 2E show the evaluation of the therapeutic effects of four different mutant strains in a unilateral tumor model.

FIG. 3 shows the efficacy evaluation of different mutant strains in the treatment of metastatic non-small cell lung cancer.

FIG. 4 shows the safety evaluation of the mutant strain U400 and a small molecule CD38 inhibitor, and the measurement of safety indexes (i.e., the body weight and the body temperature of mice) under different administration doses.

FIG. 5 shows the comparison of the efficacy of the oncolytic virus U400 in a lung cancer model under different administration doses.

FIG. 6 shows the tumor inhibitory effect of the combination of the oncolytic virus U400 and the small molecule CD38 inhibitor in a subcutaneous transplanted tumor model.

FIG. 7 shows the tumor volume changes of individual mice during treating the lung cancer models with combined administration and single-drug administration (FIG. 7A), and the comparison of the overall response rate and the comparison of the remission rate are also recorded after 30 days of continuous observation which is conducted after treatment (FIG. 7B).

FIG. 8 shows the comparison of the efficacy of combined administration and single-drug administration in controlling lung metastasis in a mouse model of metastatic lung cancer.

### DETAILED DESCRIPTION

### Definitions

When used in combination with the term “comprise” in claims and/or specification, the wording “a” or “an” may refer to “one”, but may also refer to “one or more”, “at least one” and “one or more than one”.

As used in claims and specification, the wording “comprise”, “have”, “include” or “contain” means inclusive or open-ended, and does not exclude additional and unreferenced elements, methods or steps.

Throughout the application document, the term “about” means that a value includes the standard deviation of the error of the device or method used to determine the value.

Although the definition of the term “or” as being an alternative only and as “and/or” are both supported by the disclosed content, the term “or” in claims means “and/or” unless it is explicitly indicated that the term “or” only means an alternative or the alternatives are mutually exclusive.

“Vesicular stomatitis virus (VSV)” in the present disclosure is a negative-strand RNA virus that infects most mammalian cells and expresses viral protein accounting for up to 60% of the total protein in the infected cells. In nature, VSV infects swines, cattles and horses, and causes chickenpox disease near mouth and feet. Although it has been reported that human may get infected with VSV, VSV has not caused any serious symptoms in humans. VSV encodes five kinds of proteins, including nucleocapsid protein (N), phosphoprotein (P), matrix protein (M), surface glycoprotein (G) and RNA-dependent RNA polymerase (L). Blocking the protein synthesis in the host cell by VSV matrix protein (M) may induce the death of cells.

Throughout the application document, “U400”, “virus U400”, “attenuated virus U400” or “oncolytic virus U400” refers to the following virus, the amino acid sequence encoding the modified matrix protein (M) of said virus has (i) mutation of methionine M to arginine R at position 51, (ii) mutation of valine V to phenylalanine F at position 221, and (iii) mutation of glycine G to arginine R at position 226, as compared with SEQ ID NO:1 (i.e., the amino acid sequence encoding the matrix protein of the wild-type vesicular stomatitis virus).

In a specific embodiment of the present disclosure, the sequence of the aforementioned modified matrix protein (M) is a sequence as set forth in SEQ ID NO:3.

Throughout the application document, “U000”, “virus U000”, “attenuated virus U000” or “oncolytic virus U000” refers to the following virus, the amino acid sequence encoding the modified matrix protein (M) of said virus has (i) mutation of glycine G to glutamic acid E at position 21, (ii) mutation of methionine M to alanine A at position 51, (iii) mutation of leucine L to alanine A at position 111, and (iv) mutation of valine V to phenylalanine F at position 221, as compared with SEQ ID NO:1 (i.e., the amino acid sequence encoding the matrix protein of the wild-type vesicular stomatitis virus).

Throughout the application document, “U200”, “virus U200”, “attenuated virus U200” or “oncolytic virus U200” refers to the following virus, the amino acid sequence encoding the modified matrix protein (M) of said virus has (i) mutation of methionine M to arginine R at position 51, as compared with SEQ ID NO:1 (i.e., the amino acid sequence encoding the matrix protein of the wild-type vesicular stomatitis virus).

Throughout the application document, “U500”, “virus U500”, “attenuated virus U500” or “oncolytic virus U500” refers to the following virus, the amino acid sequence encoding the modified matrix protein (M) of said virus has (i) mutation of glycine G to glutamic acid E at position 21, as compared with SEQ ID NO:1 (i.e., the amino acid sequence encoding the matrix protein of the wild-type vesicular stomatitis virus).

When used in claims and/or specification, the term “inhibition”, “reduction”, “prevention” or any variation of these terms includes any measurable reduction or complete inhibition for the purpose of achieving the desired results (for example, treatment of tumor). Desired results include but are not limited to the relief, reduction, slowing or eradication of a cancer, a hyperproliferative condition or a symptom related to a cancer, as well as the improved quality or extension of life.

In one embodiment, the present disclosure describes an attenuated rhabdovirus produced by a reverse genetic operating system, which is a novel recombinant system developed for gene therapy of tumor. An attenuated triple mutant of rhabdovirus (attenuated virus U400) has been produced, and has been demonstrated to be safe and effective in systemic delivery in a variety of tumor models (tumor models with immune function).

In one embodiment, the attenuated triple mutant of the rhabdovirus of the present disclosure (and/or other oncolytic agents) may be used continuously without causing strong immune response against the therapeutic virus in the host. Based on this, the host may be treated with the same viral system for multiple times within a certain period of time, thereby prolonging the treatment period, further reducing the occurrence of the body's resistance to a single drug and thus improving the therapeutic effects on tumor. The embodiments of the present disclosure include compositions and methods related to rhabdoviruses and the use thereof in antitumor therapy. These rhabdoviruses have the characteristic of being capable of killing tumor cells both in vivo and in vitro. In the present disclosure, the rhabdovirus may be an attenuated rhabdovirus or a genetically engineered variant of an attenuated rhabdovirus. The virus described in this application may be used in combination with other rhabdoviruses.

In one embodiment of the present disclosure, an attenuated rhabdovirus and a composition comprising the attenuated rhabdovirus are included. The sequence of said attenuated rhabdovirus encodes a variant M protein that has at least or at most 80%, 81%, 82%, 83%, 84%, 85%, 86%, 87%, 88%, 89%, 90%, 91%, 92%, 93%, 94%, 95%, 96%, 97%, 98%, 99%, 100% (including all ranges and percentages between these values) amino acid identity with the M protein of the wild-type rhabdovirus (that is, the amino acid sequence as set forth in SEQ ID NO:1). The above-mentioned M proteins of the attenuated rhabdovirus having a certain percentage of identity means that the M protein of the attenuated rhabdovirus has conservative mutations capable of normally maintaining the function of the protein. A representative example of conservative mutations is conservative substitution. Conservative substitution refers to, for example, a mutation wherein substitution takes place mutually among Phe, Trp and Tyr in a case where the substitution site is an aromatic amino acid; a mutation wherein substitution takes place mutually among Leu, Ile and Val in a case where the substitution site is a hydrophobic amino acid; a mutation wherein substitution takes place mutually between Gln and Asn in a case where the substitution site is a polar amino acid; a mutation wherein substitution takes place mutually among Lys, Arg and His in a case where the substitution site is a basic amino acid; a mutation wherein substitution takes place mutually between Asp and Glu in a case where the substitution site is an acidic amino acid; and a mutation wherein substitution takes place mutually between Ser and Thr in a case where the substitution site is an amino acid having a hydroxyl group. As substitutions considered as conservative substitutions, there may be specifically exemplified substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Gly, Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val. Furthermore, the above-mentioned mutations for the identity of the M protein of the attenuated rhabdovirus also include naturally occurring mutations which are attributed to the gene-derived individual difference, difference in strains, difference in species and the like of the rhabdovirus.

In some cases, as for individual random mutations, although each single-mutant strain may reduce the toxic effect of the virus on normal healthy cells, it is highly likely that the virus may become more toxic than the wild-type virus in tumor cells once the above-mentioned multiple sets of individual random mutations are combined. Therefore, the therapeutic index of the recombinant oncolytic rhabdovirus of the present disclosure is unexpectedly increased, which is an unexpected finding achieved based on the large-scale screening process of the attenuated strains in vitro. When multiple single-mutant attenuated strains undergo simultaneous mutations of multiple genes, most viruses lose infectivity in both tumor cells and normal cells, and a few of the viruses show virulence enhancement with enhanced cytotoxicity. It has been unexpectedly found in the present disclosure that the three amino acid mutations of the attenuated virus U400 do not cause the virulence enhancement of the virus itself, while continuously retaining the tumor-killing property. Although it has been found at cell level in vitro that the time point of the lysis of tumor cells is delayed, the specific tumor-killing property is completely retained. Meanwhile, the attenuated virus U400 does not have any toxicity to normal cells and fully meets the requirements for biosafety.

The specific meanings of the SEQ ID NOs involved in the present disclosure are as follows.

The sequence as set forth in SEQ ID NO: 1 is the amino acid sequence of the wild-type matrix protein (M) of vesicular stomatitis virus.

The sequence as set forth in SEQ ID NO: 2 is the nucleotide sequence of the wild-type matrix protein (M) of vesicular stomatitis virus.

The sequence as set forth in SEQ ID NO: 3 is the amino acid sequence of the modified matrix protein (M) of vesicular stomatitis virus.

The sequence as set forth in SEQ ID NO: 4 is the nucleotide sequence of the modified matrix protein (M) of vesicular stomatitis virus.

### EXAMPLES

Other objects, features and advantages of the present disclosure will become apparent from the following detailed description. However, it should be understood that the detailed description and specific Examples (although representing the specific embodiments of the present disclosure) are given for explanatory purposes only, since various changes and modifications made within the spirit and scope of the present disclosure will become apparent to those skilled in the art after reading this detailed description.

In the process of treating cancer (derived from LLC-T2 lung cancer cell line) by using an oncolytic rhabdovirus (for example, viruses U000, U200, U400, or U500) and a CD38 molecule inhibitor (rhein) involved in the present disclosure, the specific experimental protocols as adopted were as follows.

Formulation and administration schedule of rhein and the oncolytic virus:

1. Animals: C57BL/6 mice, female, 18 g to 20 g, 120 mice in total, purchased from Beijing Vital River Laboratory Animal Technology Co., Ltd.

2. Drugs and reagents:

2.1 Formulation of oncolytic virus: The stock solution of the oncolytic virus was diluted into a solution with a concentration of 10⁸ PFU/ml for storage.

2.2 Rhein (RH): RH powder was formulated into a yellow suspension of RH (5 mg/ml) with PBS containing 0.2% propylene glycol.

2.3 PBS buffer solution: purchased from Hyclone Company.

2.4 LLC-T2 cell line: Cells were formulated into a cell suspension (1×10⁶ cells/ml).

3. Establishment of animal tumor model

3.1 LLC unilateral tumor model

Three days after the newly arrived mice were acclimated to the environment, the right side of the back of the mice was shaved, and then 200 μl of LLC-T2 cells (1×10⁶ cells/ml, 2×10⁵ cells in total) were subcutaneously injected. Tumor volume was expected to meet the requirements of grouping 9 to 10 days after cell inoculation.

4. Groups and Administration

On Day 9 to Day 10 of the experiment, mice with a tumor volume up to approximately 100 mm³ (80 to 120 mm³) were randomly divided into groups. The whole experiment lasted for 28 days, and the groups and the dosing information were as follows.

1) PBS group: 20 mice were each administered with 200 μl of PBS buffer solution via intratumoral injection and intraperitoneal injection, wherein the intratumoral injection was given once every two days, the intraperitoneal injection was given once every four days, and both of them were given three times.

2) RH group: 20 mice were each administered with 200 μl of RH (50 mg/kg) via intraperitoneal injection once every four days for three times in total.

3) Oncolytic virus administration group: 20 mice were each administered with 100 μl of the oncolytic virus (1×10⁷ PFU) via intratumoral injection once every two days for three times in total.

4) Combination therapy (RH+oncolytic virus): 20 mice were each administered with 200 μl of RH (10 mg/kg/30 mg/kg/50 mg/kg) via intraperitoneal injection once every four days for three times in total, and were each administered with 100 μl of the oncolytic virus (1×10⁷ PFU) via intratumoral injection once every two days for three times in total.

5. Measurement of tumor volume

The long diameter and short diameter of tumor were measured with a vernier caliper every two days, and the tumor volume was calculated according to the calculation formula (as below).

Calculation formula: tumor volume (TV, mm³)=(D_{long diameter}×D_{short diameter}²)/2

6. Survival rate and survival curve

The survival rate of mice in each group was observed and recorded every day during the experiment, and the survival curves of different groups were plotted after the experiment.

7. Weighing of tumor

At the end of the experiment, mice were sacrificed, then tumors were incised and weighed using an electronic balance, and the weights were recorded.

8. Fluorescence images showing lung metastasis and in vivo imaging in small animals

At the end of the experiment, mice were sacrificed, and then the lung tissues of the mice were incised, washed with PBS buffer solution and placed in a 12-well plate. Images were taken under green light, and the red fluorescence protein appeared yellow under this light conditions. Afterwards, the proportion of the red fluorescence caused by the metastasis of cancer cells in lung tissue was quantified by using a fluorescence microscope, and a column diagram showing the proportion of the fluorescence caused by lung metastasis was plotted. If an animal died of the tumor burden, the proportion of metastasis in lung tissue was recorded as 100%.

9. Data processing

9.1 Individual tumor volume growth curve

According to the tumor volume measured at each time point, the growth curve illustrating the tumor volume change of individual mouse over time was plotted. One curve was plotted for each group, and the data derived from the left side and the right side were plotted separately. If the animal died, the final data point was marked in red.

9.2 Waterfall plot of the tumor volume change rate in the middle stage and the tumor volume change rate at the end of the experiment

Tumor volume change rates on Day 9 (the middle stage of the experiment) and on Day 18 (the end of the experiment) were calculated according to the calculation formula and waterfall plots were plotted. One curve was plotted for each group, and the data derived from the left side and the right side were plotted separately. If the animal died, the change rate was recorded as 7000 (the maximum rate).

tumor volume change rate=((final volume−initial volume)/initial volume)×100%. Calculation formula:

### Example 1: Experimental Protocol of Treating Unilateral LLC-T2 Tumor with a Combination of Rhein and Virus U400

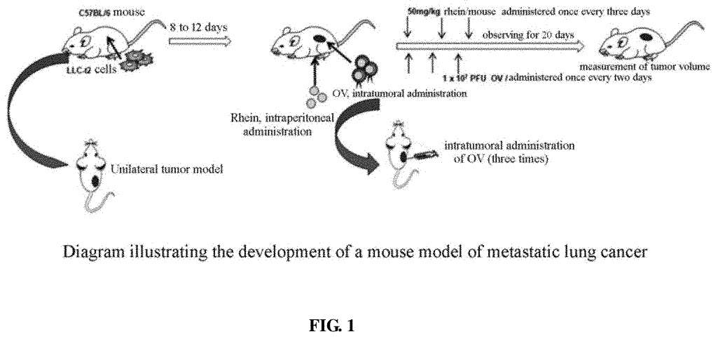

The specific implementation process was as shown in FIG. 1. First, 120 female C57BL/6 mice (18 g to 20 g) were purchased. On Day 0, each mouse was inoculated with LLC-T2 tumor cells (2×10⁵ cells) on the right side of the back. On Day 9 to Day 10, 100 tumor-bearing mice with a tumor volume of approximately 100 mm³ (80 mm³ to 120 mm³) were randomly divided into 4 groups, that is, PBS group, RH group, virus U400 group, and combination therapy (RH+virus U400) group. RH was administered via intraperitoneal injection once every four days for three times in total, and virus U400 was administered via intratumoral injection once every two days for three times in total. Tumors were measured once every two days during the experiment. On Day 29 to Day 30 of the experiment, all mice were sacrificed, and tumors were incised and weighed. After dissection, the lung tissues of the mice were incised and a fluorescence microscope was utilized to take fluorescence images illustrating the metastasis of lung cancer cells. After the completion of the experimental operation, in each group, the individual tumor growth curves and the waterfall plot of the tumor growth rate in the middle stage and the tumor growth rate at the end stage of the experiment were plotted.

### Example 2: Establishment of a Subcutaneous Transplanted Tumor Model of Lung Cancer and Comparison of Therapeutic Effects of Different Mutant Viral Strains in the Treatment of Subcutaneous Transplanted Tumor

On Day 0, 40 female C57BL/6 mice (18 g to 20 g) were each inoculated with LLC-T2 tumor cells (2.2×10⁵ cells) bilaterally on the back. On Day 9 to Day 10, the tumor-bearing mice with a tumor volume of approximately 100 mm³ (80 mm³ to 120 mm³) were randomly divided into 5 groups, that is, PBS group, U000 group, U200 group, U500 group and U400 group, and were administered three times in total. The virus was administered via intratumoral injection once every two days for three times in total. Tumors were measured once every two days during the experiment. On Day 29 to Day 30 of the experiment, all mice were sacrificed, and tumors were incised and weighed.

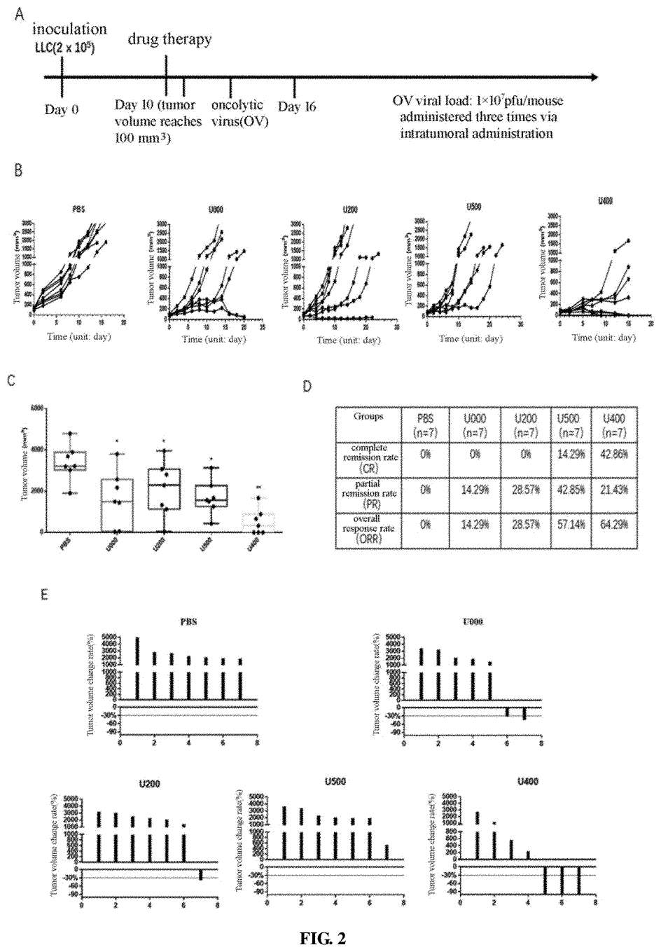

FIG. 2A showed the dosing regimen of oncolytic virus (OV) (intratumoral injection). As shown in the figure, when the tumor volume reached 100 mm³, the oncolytic virus was administered via intratumoral injection once every two days for three times in total, the administration dose was 1×10⁷ PFU, and the specific time point of the initial administration depended on the actual tumor growth rate (approximately 9 to 10 days after the inoculation of tumor cells). Based on the above-mentioned administration procedure, the therapeutic effects of different mutant strains on lung cancer were further observed and compared in mice bearing transplanted tumor derived from lung cancer.

FIG. 2B showed the curves illustrating the tumor volume change of the transplanted tumor derived from lung cancer in individual mouse over time after the intratumoral administration of different mutant strains. It was found by further comparison that, as compared with PBS group, except for U500, each of U000, U200 and U400 had certain therapeutic effect on the transplanted tumor derived from lung cancer. In the later stage of the experiment, only two mice in U000 group (n=7) and only one mouse in U200 group (n=7) had transplanted tumor with relatively small volume, while four mice in U400 group (n=7) had transplanted tumor with relatively small volume. It could be seen that the therapeutic effect of U400 on lung cancer was significantly superior to that of other mutant strains.

Further, when subjecting the tumor growth rate and the tumor volume of each mouse at the end of the experiment to statistical analysis, it was found that, except for three mice in U400 group that were completely cured, none of the mice in other groups were completely cured, the overall response rate of U400 was up to 64.29%, which was significantly superior to that of other treatment groups, and U400 had a significant therapeutic advantage (FIG. 2C and FIG. 2E).

### Example 3: Comparison of the Effects of Different Mutant Viral Strains on the Lung Metastasis of Lung Cancer

On Day 0, a transplanted tumor model of lung cancer was established in mouse by subcutaneous injection of LLC lung cancer cells (2×10⁵ cells/mouse). On Day 9, when the tumor volume reached approximately 100 mm³, mice were randomly divided into groups. Different mutant viruses were administered via intratumoral injection. At the end of the experiment, the lung tissues of all mice were incised. Since the red fluorescence protein was introduced into LLC cells, fluorescence images illustrating lung metastasis were taken under a microscope with a magnification of 40×, and the fluorescence ratios were calculated.

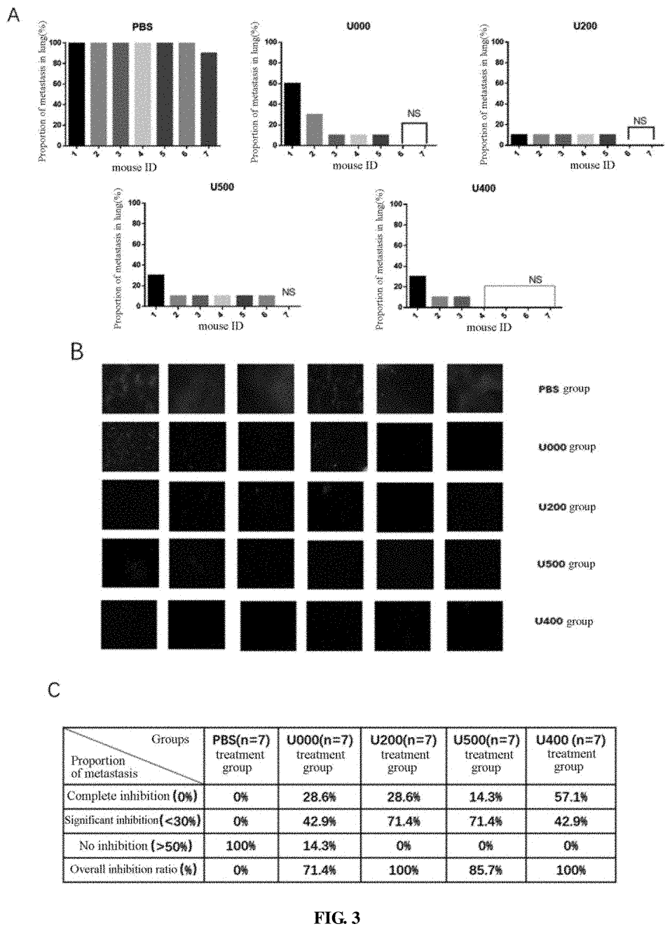

The experimental results were as shown in FIG. 3. To be specific, as shown in FIG. 3A, the proportion of metastasis in lung of each mouse in PBS group was approximately 100%, while this proportion in other treatment groups was significantly lower than that in PBS group. The ratios of complete inhibition in U000 group, U200 group and U500 group were respectively 28.6%, 28.6% and 14.3%, which were significantly lower than that in U400 group (57.1%). In U400 group, there were four mice that did not have any metastasis in lung, and the proportion of metastasis in lung of each of the remaining three mice was lower than 30%. It could be seen that the inhibitory effect of U400 on the metastasis of lung cancer cells was significantly superior to that of other mutant viral strains (FIG. 3B to FIG. 3C).

### Example 4: Safety Assessment of Single Drug (Rhein and U400) in Tumor-Bearing Mice

A transplanted tumor model of lung cancer was established in C57BL/6 mice. Different doses of U400 and rhein were administered, and the administration safety of U400 (10⁷ PFU, 10⁶ PFU or 10⁵ PFU) and the administration safety of rhein (with an administration dose of 50 mg/kg, 30 mg/kg or 10 mg/kg) in tumor-bearing mice were explored by monitoring body weight, body temperature and clinical symptoms.

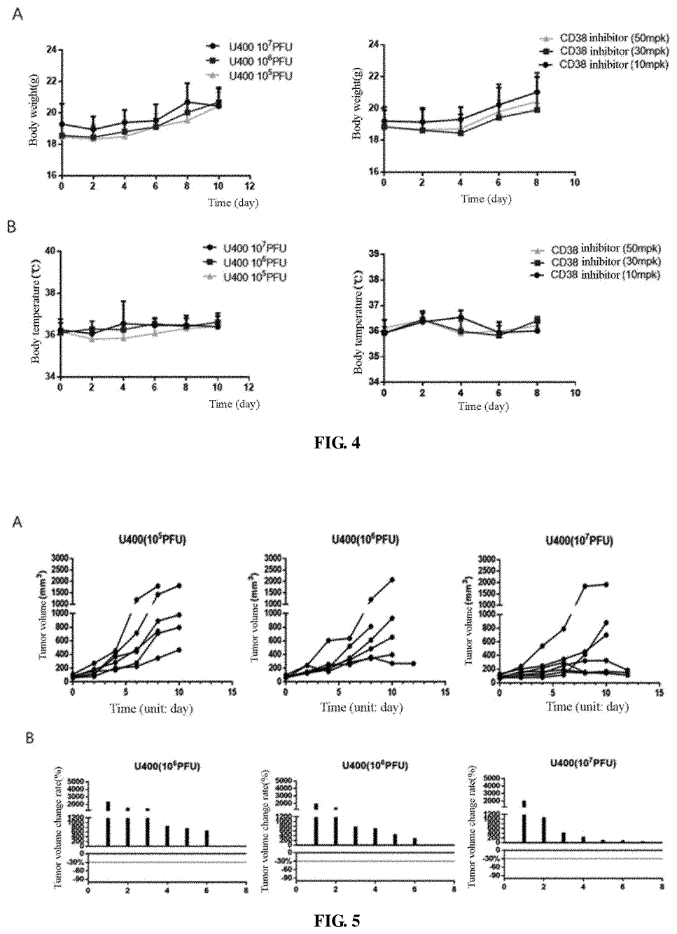

The experimental results were as shown in FIG. 4. To be specific, FIG. 4A showed the influences on the body weight of the tumor-bearing mice after the administration of different doses of U400 or rhein. As shown in the figure, abnormal influences on the body weight of the tumor-bearing mice caused by U400 or rhein were not observed during the administration period. The average body weight of the mice in each group increased slowly over time, which was consistent with the body weight change of the tumor-bearing mice. FIG. 4B showed the influences on the body temperature of the tumor-bearing mice after the administration of different doses of U400 or rhein. The administration of different doses of U400 did not exert abnormal influence on the body temperature of the tumor-bearing mice. Although the administration of different doses of rhein caused some fluctuations in the body temperature of the tumor-bearing mice, no dosage dependence was observed among each dosage group and the amplitude of changes was relatively small, and such fluctuations belonged to the harmless effects related to rhein. It was found by carefully observing the tumor-bearing mice daily during the experiment that, after the administration of rhein (50 mg/kg or 30 mg/kg), the tumor-bearing mice would have clinical symptoms of decreased activity and mental sluggishness within a short period of time and could return to the normal state after about 30 min, while no related clinical symptom was observed after the administration of rhein at a dose of 10 mg/kg.

In summary, it could be seen that different doses of U400 and rhein did not cause toxic damage to the tumor-bearing mice under the experimental conditions.

### Example 5: Exploration of the Optimal Therapeutic Dose of Single Drug (Rhein and U400) in Mice Suffering from Lung Cancer

40 female C57BL/6 mice were subcutaneously inoculated with LLC lung cancer cells. The administration was started when the tumor volume reached 100 mm³. The three doses of U400 were 10⁵ PFU, 10⁶ PFU and 10⁷ PFU, respectively. The three doses of rhein were 10 mg/kg, 30 mg/kg and 50 mg/kg, respectively. U400 was administered once every two days for three times in total, and rhein was administered once every three days for three times in total. The volume of the transplanted tumor of the mice suffering from lung cancer was measured every two days, and all mice were euthanized after being measured five times or six times.

FIG. 5A showed the effects of different doses of U400 on the volume of the transplanted tumor derived from lung cancer. As shown in the figure, the oncolytic virus U400 (10⁵ PFU or 10⁶ PFU) had poor tumor inhibitory effects on mice suffering from lung cancer, and 10⁷ PFU of U400 was capable of significantly inhibiting the tumor volume of the transplanted tumor of mice suffering from lung cancer. Further, according to the growth rate of tumor volume of each mouse at the end of the experiment (FIG. 5B), it could be seen that, three mice in the U400 group (with an administration dose of 10⁷ PFU) had a tumor growth rate lower than 200%, while the therapeutic effects were poor at the other two dose levels. Based on the results of the safety assessment and the therapeutic effects, it could be seen that the therapeutic effect of U400 increased with the increase of dose and were dose-dependent to a certain extent.

In summary, the same conclusion could be seen from FIG. 5. Based on the results of the safety assessment, it could be seen that the impact on mice was relatively small at a dose of 10 mg/kg. Therefore, it could be known that the optimal dose of rhein was 10 mg/kg.

### Example 6: Exploration of the Therapeutic Effect of Combined Administration of Rhein and U400 in Mice Suffering from Lung Cancer

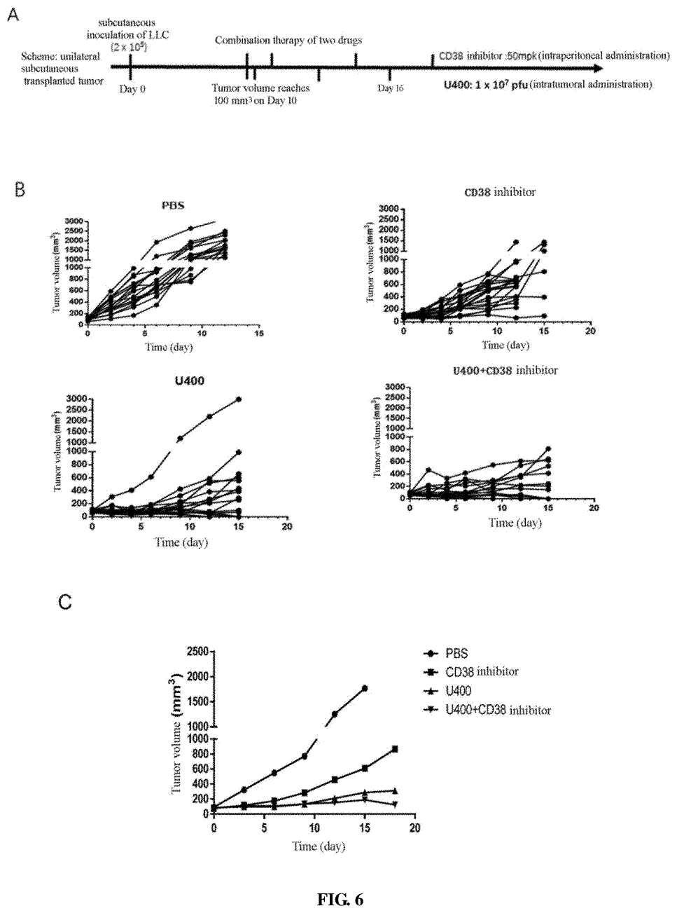

FIG. 6A showed the dosing regimen of the combination therapy of the oncolytic virus U400 and the CD38 inhibitor (i.e., rhein). On Day 0 of the experiment, a transplanted tumor model of lung cancer was established by subcutaneous injection of LLC cells. On Day 10, when the tumor volume reached 80 mm³ to 100 mm³, the tumor-bearing mice that met the requirements were grouped and administered. The oncolytic virus U400 was administered once on Day 10, Day 12 and Day 14 via intratumoral injection at a dose of 1×10⁷ PFU/mice, and rhein was administered once on Day 10, Day 13 and Day 16 via intraperitoneal injection at a dose of 50 mg/kg. During the experiment, the volume of the transplanted tumor in mice was measured once every two days.

The experimental results were as shown in FIG. 6B. The increase of the tumor volume of the tumor-bearing mice in PBS group was normal, and certain therapeutic effects on tumor were shown in the rhein group, the U400 group and the combination therapy group (U400+rhein). In the later stage of the experiment, the tumor volumes of the mice in the U400 group and the combination therapy group were all 1000 mm³ or less, which was significantly superior to the therapeutic effect achieved by the rhein group. Further, according to the growth curves of the average tumor volume of different groups (FIG. 6C), it could be seen that the effect achieved by the combination therapy group was significantly superior to that achieved by the rhein group and the U400 group (monotherapy group) and the tumor volumes in the combination therapy group were small at the end of the experiment.

### Example 7: Comparison of the Inhibitory Effect of the Combined Administration of Rhein and U400 and the Inhibitory Effect of Monotherapy in Lung Metastasis Model

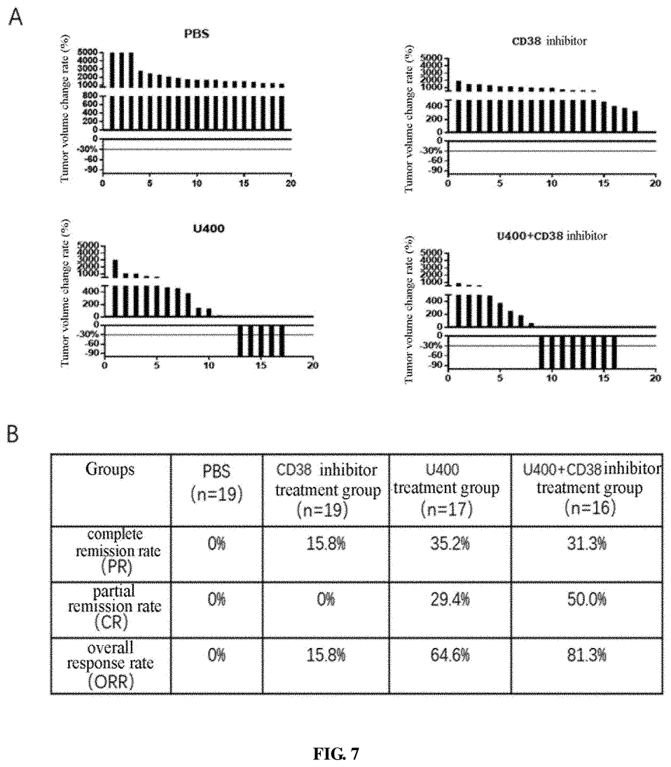

FIG. 7A showed the statistical results of the therapeutic effect of the combination therapy of the oncolytic virus U400 and the CD38 inhibitor (i.e., rhein) o metastatic lung cancer. It could be found that, complete remission was achieved in approximately 50% of the mice and the overall response rate increased to 81% in the combination therapy group, while complete remission was achieved in only 29% of the mice in U400 administration group and none of the mice treated with rhein alone showed complete remission. As shown in FIG. 7B, the statistical results of the evaluation of the therapeutic effects in metastatic lung cancer mouse model indicated that the combination of U400 and rhein produced a synergistic therapeutic effect, and as compared with the monotherapy groups, the combined administration group had significant advantage in terms of controlling tumor growth, especially killing tumors completely.

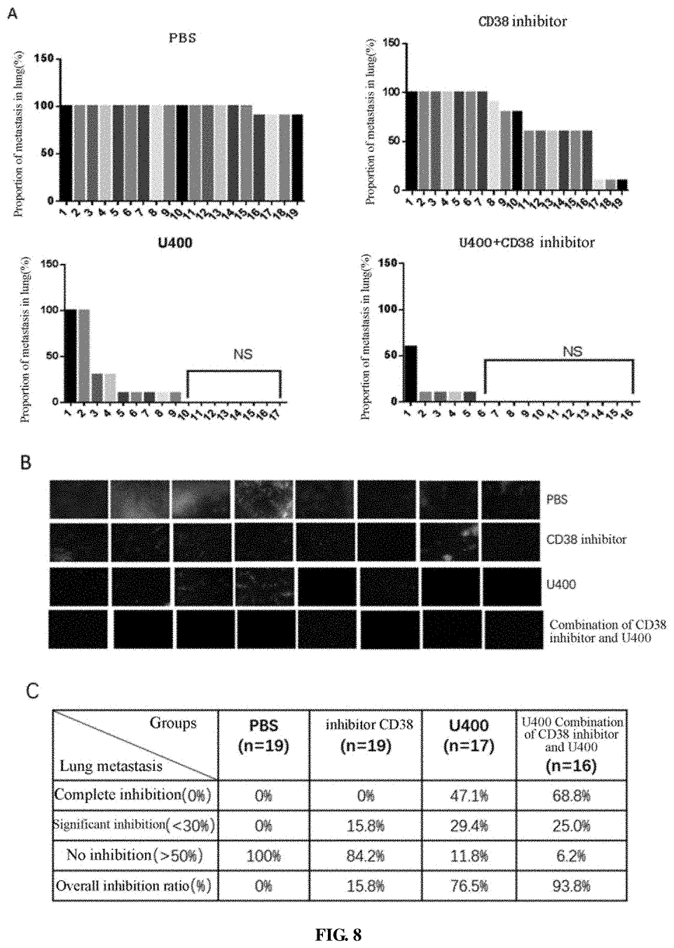

The lung cancer mouse models were further administered with PBS, rhein, U400 and a combined administration of rhein and U400. After the completion of the experiment, the lung metastases of the mice were observed and evaluated. As could be seen from FIG. 8A and FIG. 8B, lung cancer cells almost occupied the lung tissue of the mice and the proportion of metastasis was approximately 100% in PBS group, while lung metastasis was effectively controlled in the U400 group and the combination therapy group. The ratio of complete inhibition was 68.8% in the combination therapy group, which was significantly superior to that in the U400 group (47.1%). From the perspective of the overall inhibition ratio, the overall inhibition ratio was 93.8% in the combination therapy group, which was significantly superior to those in the U400 group (76.5%) and the rhein group (15.8%). As could be seen from the control of the tumor volumes of the LLC lung cancer mouse models in each treatment group in Example 6, the therapeutic effect on lung cancer and the inhibitory effect on the metastasis of lung cancer cells in the combination therapy group (U400+rhein) were significantly superior to those in the U400 or rhein monotherapy group. Further, the response rates of all tumor-bearing mice were subjected to statistical analysis. As shown in FIG. 8C, a total of 16 mice suffering from cancer were enrolled in the combination therapy group and the overall inhibition ratio reached 93.8%, which was much higher than that in the administration group where the small molecule CD38 inhibitor was administered alone. Therefore, it was further proved that the combined administration of the CD38 inhibitor and U400 significantly improved the overall response rate of mice and improved the survival rate of mice. Meanwhile, the cure rate in the combination therapy group reached 68.8%, which was significantly higher than that of the U400 monotherapy group.

The above-mentioned Examples of the present disclosure are merely exemplified to clearly illustrate the present disclosure rather than limitations to the embodiments of the present disclosure. For those of ordinary skill in the art, other changes or modifications in different forms may also be made based on the foregoing description. It is not necessary and impossible to enumerate all the embodiments. Any modification, equivalent replacement and improvement made within the spirits and principles of this disclosure shall be encompassed in the protection scope of the claims of the present disclosure.

1. A composition comprising (a) an oncolytic rhabdovirus and (b) a CD38 molecule inhibitor.

2. (canceled)

3. The composition according to claim 1, wherein the oncolytic rhabdovirus comprises a modified matrix protein (M), an amino acid sequence encoding the modified matrix protein (M) has at least 80% identity with an amino acid sequence as set forth in SEQ ID NO: 1; and
the amino acid sequence has amino acid substitutions at position 51, position 221 and position 226 as compared with SEQ ID NO: 1.

4. The composition according to claim 3, wherein the sequence of the modified matrix protein (M) is the amino acid sequence encoding the modified matrix protein (M) and has the following mutations as compared with SEQ ID NO:1:
(i) mutation of methionine M to arginine R at position 51,
(ii) mutation of valine V to phenylalanine F at position 221, and
(iii) mutation of glycine G to arginine R at position 226.

5. The composition according to claim 1, wherein the CD38 molecule inhibitor is selected from a combination comprising one or more selected from rhein and its analogs.

6. The composition according to claim 5, wherein active substances in the composition further comprise one or more selected from a combination of other active substances that control or treat a tumor, wherein said other active substance is selected from clofibrates, choline, methionine, niacin-based substances and ursodeoxycholic acid.

7. The composition according to claim 6, wherein the composition further comprises a second oncolytic virus.

8. The composition according to claim 7, wherein the composition further comprises a second antitumor preparation.

9. (canceled)

10. (canceled)

11. The composition according to claim 1, wherein the oncolytic rhabdovirus and the CD38 molecule inhibitor are each independently present in the composition without being mixed with each other.

12. The composition according to claim 1, wherein the oncolytic rhabdovirus is a genetically mutated attenuated strain having oncolytic effect or a wild-type virus having oncolytic effect.

13. (canceled)

14. (canceled)

15. (canceled)

16. (canceled)

17. (canceled)

18. A method for inhibiting and/or killing abnormally proliferating cells in a subject, the method comprising sequentially carrying out the following steps in the subject:
1) administering an oncolytic rhabdovirus to the subject, wherein the oncolytic rhabdovirus is capable of replicating in tumor cells selectively; and
2) administering a CD38 molecule inhibitor to the subject after administration of the oncolytic rhabdovirus in step 1);
alternatively, administering the CD38 molecule inhibitor to the subject 24 hours to 48 hours after the administration of the oncolytic rhabdovirus.

19. The method according to claim 18, wherein the CD38 molecule inhibitor is selected from a combination comprising one or more selected from rhein and its analogs; and
the oncolytic rhabdovirus comprises a modified matrix protein (M), an amino acid sequence encoding the modified matrix protein (M) has at least 80% identity with an amino acid sequence as set forth in SEQ ID NO: 1; and
the amino acid sequence has amino acid substitutions at position 51, position 221 and position 226 as compared with SEQ ID NO: 1.

20. (canceled)

21. (canceled)

22. The method according to claim 18, wherein the oncolytic rhabdovirus, a composition comprising an isolated recombinant oncolytic rhabdovirus, or a vaccine comprising an isolated recombinant oncolytic rhabdovirus is administered via one or more administration modes selected from the group consisting of intraperitoneal administration, intravenous administration, intraarterial administration, intramuscular administration, intradermal administration, intratumoral administration, subcutaneous administration and intranasal administration.

23. The method according to claim 18, wherein the abnormally proliferating cells are selected from tumor cells and/or cancer cells.

24. The method according to claim 18, wherein the method further comprises a step of administering a second antitumor therapy.

25. (canceled)

26. (canceled)

27. (canceled)

28. A method for inducing immune response in a subject, wherein the method comprises administering the composition of claim 1 to the subject.

29. The method according to claim 28, wherein the CD38 molecule inhibitor in the composition is selected from a combination comprising one or more selected from rhein and its analogs; and
the oncolytic rhabdovirus comprises a modified matrix protein (M), an amino acid sequence encoding the modified matrix protein (M) has at least 80% identity with an amino acid sequence as set forth in SEQ ID NO: 1; and
the amino acid sequence has amino acid substitutions at position 51, position 221 and position 226 as compared with SEQ ID NO: 1.

30. The method according to claim 28, comprising sequentially carrying out the following steps in the subject:
1) administering the oncolytic rhabdovirus to the subject, wherein the oncolytic rhabdovirus is capable of replicating in tumor cells selectively; and
2) administering the CD38 molecule inhibitor to the subject after administration of the oncolytic rhabdovirus in step 1);
alternatively, administering the CD38 molecule inhibitor to the subject 24 hours to 48 hours after the administration of the oncolytic rhabdovirus.

31. A method for inducing and promoting antitumor immune response or eliminating immunosuppression in a microenvironment of a tumor tissue, wherein the method comprises a step of contacting a tumor or a tumor tissue with the composition of claim 1.

32. The method according to claim 31, wherein the CD38 molecule inhibitor is selected from a combination comprising one or more selected from rhein and its analogs; and
the oncolytic rhabdovirus comprises a modified matrix protein (M), an amino acid sequence encoding the modified matrix protein (M) has at least 80% identity with an amino acid sequence as set forth in SEQ ID NO: 1; and
the amino acid sequence has amino acid substitutions at position 51, position 221 and position 226 as compared with SEQ ID NO: 1.

33. The method according to claim 31, wherein the method comprises the following steps:
1) administering the oncolytic rhabdovirus to the subject so as to allow the tumor or the tumor tissue of the subject to contact with the oncolytic rhabdovirus, wherein the oncolytic rhabdovirus is capable of replicating in tumor cells selectively; and
2) after administration of the oncolytic rhabdovirus in step 1), administering the CD38 molecule inhibitor to the subject so as to allow the tumor or the tumor tissue of the subject to contact with the CD38 inhibitor;
alternatively, administering the CD38 molecule inhibitor to the subject 24 hours to 48 hours after the administration of the oncolytic rhabdovirus.
